(19) United States
(12) Patent Application Publication
(10) Pub. No.: US 2023/0256096 A1
(43) Pub. Date: Aug. 17, 2023

(54) STABILIZING AGENT FOR PROBIOTIC COMPOSITION

(71) Applicant: Omya International AG, Oftringen (CH)

(72) Inventors: Lalit SHARMA, Zofingen (CH); Tanja BUDDE, Brittnau (CH)

(73) Assignee: Omya International AG, Oftringen (CH)

(21) Appl. No.: 18/004,742

(22) PCT Filed: Jul. 20, 2021
(86) PCT No.: PCT/EP2021/070233

§ 371 Date: Jan. 9, 2023

(30) Foreign Application Priority Data
Jul. 20, 2020 (EP) 20186782.7

(51) Int. Cl. A61K 47/02; A61K 35/747; A23L 33/135; A61K 8/19

(52) U.S. Cl. CPC A61K 47/02; A61K 35/747; A23L 33/135; A61K 8/19; A61K 2035/115

(57) ABSTRACT

The present invention relates to the use of surface-reacted calcium carbonate as stabilizing agent for a probiotic composition, wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source.

The present invention relates to the field of probiotics, and in particular to the use of surface-reacted calcium carbonate as stabilizing agent for a probiotic composition, as well as a method for stabilizing a probiotic microorganism culture, and a process for preparing a dry stabilized probiotic composition.

Probiotics are live microorganisms, which when administered in adequate amounts confer a health benefit on the host. Microorganisms used as probiotics are derived from different genera and species and have been studied for a variety of health and disease endpoints. Currently, both yeast and bacteria are used as probiotics, including lactic acid bacteria, Bifidobacterium, Propionibacterium, Bacillus and Escherichia coli. They may be naturally occurring microorganisms, or microorganisms that have been genetically altered for a specific effect. (cf. Sanders et al., Gut Microbes 2010, 1, 3, 164-185).

Probiotics are naturally present in fermented foods, may be added to other food products, and are available as dietary supplements. Probiotics are identified by their specific strain, which includes the genus, the species, the subspecies (if applicable), and an alphanumeric strain designation. The seven core genera of microbial organisms most often used in probiotic products are Lactobacillus, Bifidobacterium, Saccharomyces, Streptococcus, Enterococcus, Escherichia, and Bacillus.

Probiotics exert their effects usually in the gastrointestinal tract, where they may influence the intestinal microflora, the activity and composition of which can affect human health and disease. It was found that probiotics can transiently colonize the human gut mucosa in highly individualized patterns, depending on the baseline microbiota, probiotic strain, and gastrointestinal tract region.

Furthermore, probiotics may exert health effects by nonspecific, species-specific, and strain-specific mechanisms. These mechanisms include inhibition of the growth of pathogenic microorganisms in the gastrointestinal tract, e.g., by fostering colonization resistance, improving intestinal transit, or helping normalize a perturbed microbiota, production of bioactive metabolites, and reduction of luminal pH in the colon. Species-specific mechanisms can include vitamin synthesis, gut barrier reinforcement, bile salt metabolism, enzymatic activity, and toxin neutralization. Strain-specific mechanisms, which are rare and are used by only a few strains of a given species, include cytokine production, immunomodulation, and effects on the endocrine and nervous systems. Through all of these mechanisms, probiotics might have wide-ranging impacts on human health and disease.

The live microorganisms used to make many fermented foods, including yogurt, typically survive well in the product throughout its shelf life. However, they usually do not survive transit through the stomach and might not resist degradation in the small intestine by hydrolytic enzymes and bile salts and, therefore, might not reach the distal gut. However, legitimate probiotic strains contained in yogurt or other foods do survive intestinal transit.

Probiotics are also available as dietary supplements (in capsules, powders, liquids, and other forms) containing a wide variety of strains and doses. These products often contain mixed cultures of live microorganisms rather than single strains. The concentration of the probiotic microorganisms in probiotic compositions is typically measured in colony forming units (CFU), which indicate the number of viable cells. Many probiotic supplements contain 10⁹ to 10¹⁰ CFU per dose, but some products contain up to 5×10¹⁰ CFU or more. However, higher CFU counts do not necessarily improve the product's health effects. Because probiotics must be consumed alive to have health benefits and they can die during their shelf life, the CFU number at the time of manufacture is not meaningful, but the CFU number at the end of the product's shelf life (cf. National Institutes of Health, Probiotics, Fact Sheet for Health professionals).

Bulk probiotics are typically prepared by adding stabilizers such as poly- or oligosaccharides to concentrates from fermentation vessels, and freeze or spray drying said concentrations afterwards, as described in US20050100559. The obtained dry material may then be milled into the powder. However, it is challenging to produce probiotic composition that have a reasonable long shelf-life, in particular at room temperature, i.e. they retain a concentration of at least 10⁶ viable cells (colony forming units, CFU) per gram of the formulation.

WO2010054439 A1 discloses a probiotic composition comprising a probiotic microorganism embedded within a matrix, wherein said matrix maintains the viability of said microorganism.

EP3520798 A1 relates to a dosage form comprising functionalized calcium carbonate serving as active ingredient, preferably for the release of calcium. A carrier for the controlled release of active agents, comprising a core, comprising surface-reacted natural or synthetic calcium carbonate, and at least one active agent being associated with said surface-reacted calcium carbonate, and a coating encapsulating the core is described in WO2013068478 A1.

However, there is still a need in the art for further methods for stabilizing probiotic microorganisms, and especially probiotic compositions having an extended shelf life.

Accordingly, it is an object of the present invention to provide a stabilizing agent for a probiotic composition. It would be desirable that the stabilizing agent reduces or prevents degradation of probiotic microorganisms during drying of a probiotic composition and/or extends shelf-life of a probiotic composition. It would also be desirable that the stabilizing agent is derivable from natural resources, is environmentally safe, and easy degradable.

It is also an object of the present invention to provide a probiotic composition having an extended shelf-live, in particular at room temperature. It is also desirable that the probiotic composition is suitable for consumption by people with special dietary requirements, such as infants, young children, elderly people, or diabetic patients. For example, it would be desirable that the probiotic composition does not include sugars, polysaccharides, or synthetic encapsulation materials.

According to one aspect of the present invention, use of surface-reacted calcium carbonate as stabilizing agent for a probiotic composition is provided, wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source.

According to a further aspect of the present invention, a method for stabilizing a probiotic microorganism culture is provided, comprising the step of mixing a probiotic microorganism culture with a surface-reacted calcium carbonate in an aqueous medium, wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source, and drying the obtained mixture, wherein preferably the drying is carried out by spray drying, freeze drying, flash drying, fluidized bed drying, jet drying, vacuum drying, or a combination thereof.

According to a further aspect of the present invention, a process for preparing a dry stabilized probiotic composition is provided, comprising the steps of:

a) providing an aqueous probiotic composition comprising at least 75 wt.-% of a probiotic microorganism culture, based on the total weight of the probiotic composition,

b) providing an aqueous suspension comprising 10 to 30 wt.-%, based on the total weight of the aqueous suspension, of surface-reacted calcium carbonate, wherein the surface-reacted calcium carbonate is a reaction product of natural ground calcium carbonate or precipitated calcium carbonate with carbon dioxide and one or more H₃O⁺ ion donors selected from the group consisting of hydrochloric acid, sulphuric acid, sulphurous acid, phosphoric acid, oxalic acid, or mixtures thereof, wherein the carbon dioxide is formed in situ by the H₃O⁺ ion donors treatment and/or is supplied from an external source,

wherein the surface-reacted calcium carbonate has a volume median particle size d₅₀ from 0.1 to 75 μm, and a volume top cut particle size d₉₈ from 0.2 to 150 μm, a specific surface area of from 15 m²/g to 200 m²/g, measured using nitrogen and the BET method, and

wherein the weight ratio of probiotic microorganism culture:surface-reacted calcium carbonate is from 5:95 to 40:60,

c) mixing the probiotic composition of step a) and the surface-reacted calcium carbonate of step b), and

d) spray drying the mixture obtained in step c), at an inlet temperature from 130 to 210° C. and an outlet temperature from 50 to 130° C.

According to still a further aspect of the present invention a dry stabilized probiotic composition obtainable by the process according to the present invention is provided.

According to still a further aspect of the present invention, a product comprising the dry stabilized probiotic composition according to the present invention is provided, wherein the product is a tablet, a capsule, a chewable tablet, a chewable gum, a chewable pastille, a lozenge, a powder, a granulate, a pellet, a paste, a cream, a food, a feed, or a beverage.

According to still a further aspect of the present invention, use of a dry stabilized probiotic composition according to the present invention in pharmaceutical, nutritional or cosmetic applications is provided.

Advantageous embodiments of the present invention are defined in the corresponding subclaims.

According to one embodiment of the present invention, the probiotic composition comprises a probiotic microorganism culture selected from the group consisting of Bifidobacterium adolescentis, Bifidobacterium lactis, Bifidobacterium infantis, Bifidobacterium longum, Bifidobacterium bifidum, Bifidobacterium breve, Lactobacillus acidophilus, Lactobacillus johnsonii, Lactobacillus casei, Lactobacillus reuteri, Lactobacillus rhamnosus, Lactobacillus plantarum, Lactococcus lactis, Enterococcus faecium, Escherichia coli Nissle 1917, Escherichia coli criodesiccata (O83:K24:H31), Saccharomyces boulardii, Saccharomyces cerevisiae, and mixtures thereof.

According to another embodiment of the present invention, the probiotic composition comprises a probiotic microorganism culture in an amount of at least 50 wt.-%, based on the total weight of the probiotic composition, more preferably in an amount of at least 75 wt.-%, even more preferably in an amount of at least 90 wt.-%, even more preferably in an amount of at least 95 wt.-%, and most preferably the probiotic composition consists of the probiotic microorganism culture.

According to another embodiment of the present invention, the probiotic composition is a dry composition or an aqueous suspension, and preferably the probiotic composition is a dry composition.

According to another embodiment of the present invention, the surface-reacted calcium carbonate has a volume median particle size d₅₀ from 0.1 to 75 μm, preferably from 0.5 to 50 μm, more preferably from 1 to 40 μm, even more preferably from 1.2 to 30 μm, and most preferably from 1.5 to 15 μm, and/or a volume top cut particle size d₉₈ from 0.2 to 150 μm, preferably from 1 to 100 μm, more preferably from 2 to 80 μm, even more preferably from 2.4 to 60 μm, and most preferably from 3 to 30 μm, and/or a specific surface area of from 15 m²/g to 200 m²/g, preferably from 20 m²/g to 180 m²/g, more preferably from 25 m²/g to 140 m²/g, even more preferably from 27 m²/g to 120 m²/g, and most preferably from 30 m²/g to 100 m²/g, measured using nitrogen and the BET method, and/or an intra-particle intruded specific pore volume in the range from 0.1 to 2.3 cm³/g, preferably from 0.2 to 2.0 cm³/g, more preferably from 0.3 to 1.8 cm³/g, and most preferably from 0.35 to 1.6 cm³/g, calculated from mercury porosimetry measurement.

According to another embodiment of the present invention, the natural ground calcium carbonate is selected from the group consisting of marble, chalk, limestone, and mixtures thereof, or the precipitated calcium carbonate is selected from the group consisting of precipitated calcium carbonates having an aragonitic, vateritic or calcitic crystal form, and mixtures thereof, and/or the at least one H₃O⁺ ion donor is selected from the group consisting of hydrochloric acid, sulphuric acid, sulphurous acid, phosphoric acid, citric acid, oxalic acid, an acidic salt, acetic acid, formic acid, and mixtures thereof, preferably the at least one H₃O⁺ ion donor is selected from the group consisting of hydrochloric acid, sulphuric acid, sulphurous acid, phosphoric acid, oxalic acid, H₂PO₄⁻, being at least partially neutralised by a cation selected from Li⁺, Na⁺ and/or K⁺, HPO₄²⁻, being at least partially neutralised by a cation selected from Li⁺, Na⁺, K⁺, Mg²⁺, and/or Ca²⁺, and mixtures thereof, more preferably the at least one H₃O⁺ ion donor is selected from the group consisting of hydrochloric acid, sulphuric acid, sulphurous acid, phosphoric acid, oxalic acid, or mixtures thereof, and most preferably, the at least one H₃O⁺ ion donor is phosphoric acid.

According to another embodiment of the present invention, the weight ratio of probiotic microorganism culture:surface-reacted calcium carbonate is from 5:95 to 40:60, preferably from 10:90 to 35:65, more preferably from 15:85 to 30:70, and most preferably from 20:80 to 25:75.

According to another embodiment of the present invention, the stabilizing agent is a drying stabilizer and/or a shelf live preservative.

According to another embodiment of the present invention, the probiotic composition is a pharmaceutical probiotic composition, a nutritional probiotic composition, or a cosmetic probiotic composition, and/or the probiotic composition is comprised by a tablet, a capsule, a chewable tablet, a chewable gum, a chewable pastille, a lozenge, a powder, a granulate, a pellet, a paste, a cream, a food, a feed, or a beverage.

According to another embodiment of the present invention, the concentration of viable probiotic microorganism culture after drying of the probiotic composition is increased by at least 5%, preferably by at least 10%, more preferably by at least 15%, and most preferably by at least 20%, compared to a probiotic composition comprising maltodextrin as stabilizing agent.

It should be understood that for the purpose of the present invention, the following terms have the following meaning:

as used herein, the term “probiotic” refers to one or more microorganisms that confer a health benefit on a host organism, for example, a human. Examples of health benefits derived from probiotic microorganisms are reduction of pathogen load in the digestive tract, improved microbial fermentation pattern in the digestive tract, improved nutrition absorption, improved immune function, aided digestion, or relive of symptoms of irritable bowel disease and colitis.

The term “microorganism” in the meaning of the present invention refers to a single-celled organism, for example, a bacterium or a yeast.

As used herein, the term “microorganism culture” refers to a preparation of microorganisms, optionally, containing nutrients, microorganism excretions, and other soluble material present in fermentation cultures of microorganisms.

A “probiotic composition” in the meaning of the present invention is a composition comprising a probiotic microorganism culture. For example, the probiotic microorganism culture may be present in the probiotic composition in an amount of at least 10 wt.-%, based on the total amount of the probiotic composition, preferably in an amount of at least 20 wt.-%, more preferably in an amount of at least 30 wt.-%, and most preferably in an amount of at least 50 wt.-%.

“Natural ground calcium carbonate” (GCC) in the meaning of the present invention is a calcium carbonate obtained from natural sources, such as limestone, marble, or chalk, and processed through a wet and/or dry treatment such as grinding, screening and/or fractionating, for example, by a cyclone or classifier.

“Precipitated calcium carbonate” (PCC) in the meaning of the present invention is a synthesised material, obtained by precipitation following reaction of carbon dioxide and lime in an aqueous, semi-dry or humid environment or by precipitation of a calcium and carbonate ion source in water. PCC may be in the vateritic, calcitic or aragonitic crystal form. PCCs are described, for example, in EP2447213 A1, EP2524898 A1, EP2371766 A1, EP1712597 A1, EP1712523 A1, or WO2013142473 A1.

The term “surface-reacted” in the meaning of the present application shall be used to indicate that a material has been subjected to a process comprising partial dissolution of said material upon treatment with an H₃O⁺ ion donor (e.g., by use of water-soluble free acids and/or acidic salts) in aqueous environment followed by a crystallization process which may occur in the absence or presence of further crystallization additives.

An “H₃O⁺ ion donor” in the context of the present invention is a Brønsted acid and/or an acid salt, i.e. a salt containing an acidic hydrogen. The term “acid” as used herein refers to an acid in the meaning of the definition by Brønsted and Lowry (e.g., H₂SO₄, HSO₄⁻). The term “free acid” refers only to those acids being in the fully protonated form (e.g., H₂SO₄).

The “particle size” of particulate materials is described by its distribution of particle sizes dₓ. Unless indicated otherwise, the value dₓ represents the diameter relative to which x % by weight of the particles have diameters less than dₓ. This means that, for example, the d₂₀ value is the particle size at which 20 wt.-% of all particles are smaller than that particle size. The d₅₀ value is thus the weight median particle size, i.e. 50 wt.-% of all particles are smaller than this particle size. For the purpose of the present invention, the particle size is specified as weight median particle size d₅₀ (wt.) unless indicated otherwise. Particle sizes were determined by using a Sedigraph™ 5100 instrument or Sedigraph™ 5120 instrument of Micromeritics Instrument Corporation. The method and the instrument are known to the skilled person and are commonly used to determine the particle size of fillers and pigments. The measurements were carried out in an aqueous solution of 0.1 wt.-% Na₄P₂O₇.

For certain materials specified herein, the “particle size” is described as volume-based particle size distribution. This is indicated, for example, by “volume based median particle size”, “volume median particle size” or “volume top cut particle size”. Volume median particle size d₅₀ was evaluated using a Malvern Mastersizer 2000 or 3000 Laser Diffraction System. The d₅₀ or d₉₈ value, measured using a Malvern Mastersizer 2000 or 3000 Laser Diffraction System, preferably a Malvern Mastersizer 3000 Laser Diffraction System, indicates a diameter value such that 50% or 98% by volume, respectively, of the particles have a diameter of less than this value. The raw data obtained by the measurement are analysed using the Mie theory, with a particle refractive index of 1.57 and an absorption index of 0.005. The measurements were carried out in an aqueous solution of 0.1 wt.-% Na₄P₂O₇.

The term “particulate” in the meaning of the present application refers to materials composed of a plurality of particles. Said plurality of particles may be defined, for example, by its particle size distribution. The expression “particulate material” may comprise granules, powders, grains, tablets, or crumbles.

The “specific surface area” (expressed in m²/g) of a material as used throughout the present document can be determined by the Brunauer Emmett Teller (BET) method with nitrogen as adsorbing gas and by use of a ASAP 2460 instrument from Micromeritics. The method is well known to the skilled person and defined in ISO 9277:2010. Prior to such measurements, the sample was filtered within a Büchner funnel, rinsed with deionised water and dried at 110° C. in an oven for at least 12 hours. The total surface area (in m²) of said material can be obtained by multiplication of the specific surface area (in m²/g) and the mass (in g) of the material.

In the context of the present invention, the term “pore” is to be understood as describing the space that is found between and/or within particles, i.e. that is formed by the particles as they pack together under nearest neighbour contact (interparticle pores), such as in a powder or a compact and/or the void space within porous particles (intraparticle pores), and that allows the passage of liquids under pressure when saturated by the liquid and/or supports absorption of surface wetting liquids.

Unless specified otherwise, the term “drying” refers to a process according to which at least a portion of water is removed from a material to be dried such that a constant weight of the obtained “dried” material at 200° C. is reached. Moreover, a “dried” or “dry” material may be defined by its total moisture content which, unless specified otherwise, is less than or equal to 1.0 wt.-%, preferably less than or equal to 0.5 wt.-%, more preferably less than or equal to 0.2 wt.-%, and most preferably between 0.03 and 0.07 wt.-%, based on the total weight of the dried material.

For the purpose of the present application, “water-insoluble” materials are defined as those which, when mixed with 100 ml of deionised water and filtered at 20° C. to recover the liquid filtrate, provide less than or equal to 0.1 g of recovered solid material following evaporation at 95 to 100° C. of 100 g of said liquid filtrate. “Water-soluble” materials are defined as materials leading to the recovery of greater than 0.1 g of solid material following evaporation at 95 to 100° C. of 100 g of said liquid filtrate. In order to assess whether a material is an insoluble or soluble material in the meaning of the present invention, the sample size is greater than 0.1 g, preferably 0.5 g or more.

A “suspension” or “slurry” in the meaning of the present invention comprises undissolved solids and water, and optionally further additives, and usually contains large amounts of solids and, thus, is more viscous and can be of higher density than the liquid from which it is formed.

Where an indefinite or definite article is used when referring to a singular noun, e.g., “a”, “an” or “the”, this includes a plural of that noun unless anything else is specifically stated.

Where the term “comprising” is used in the present description and claims, it does not exclude other elements. For the purposes of the present invention, the term “consisting of” is considered to be a preferred embodiment of the term “comprising”. If hereinafter a group is defined to comprise at least a certain number of embodiments, this is also to be understood to disclose a group, which preferably consists only of these embodiments.

Terms like “obtainable” or “definable” and “obtained” or “defined” are used interchangeably. This, for example, means that, unless the context clearly dictates otherwise, the term “obtained” does not mean to indicate that, for example, an embodiment must be obtained by, for example, the sequence of steps following the term “obtained” though such a limited understanding is always included by the terms “obtained” or “defined” as a preferred embodiment.

Whenever the terms “including” or “having” are used, these terms are meant to be equivalent to “comprising” as defined hereinabove.

According to the present invention use of surface-reacted calcium carbonate as stabilizing agent for a probiotic composition is provided. The surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source.

In the following details and preferred embodiments of the inventive use will be set out in more details. It is to be understood that these technical details and embodiments also apply to the inventive methods, processes, compositions, and articles.

### The Surface-Reacted Calcium Carbonate

The surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source.

A H₃O⁺ ion donor in the context of the present invention is a Brønsted acid and/or an acid salt.

In a preferred embodiment of the invention the surface-reacted calcium carbonate is obtained by a process comprising the steps of: (a) providing a suspension of natural or precipitated calcium carbonate, (b) adding at least one acid having a pKa value of 0 or less at 20° C. or having a pKa value from 0 to 2.5 at 20° C. to the suspension of step (a), and (c) treating the suspension of step (a) with carbon dioxide before, during or after step (b). According to another embodiment the surface-reacted calcium carbonate is obtained by a process comprising the steps of: (A) providing a natural or precipitated calcium carbonate, (B) providing at least one water-soluble acid, (C) providing gaseous CO₂, (D) contacting said natural or precipitated calcium carbonate of step (A) with the at least one acid of step (B) and with the CO₂ of step (C), characterised in that: (i) the at least one acid of step B) has a pKa of greater than 2.5 and less than or equal to 7 at 20° C., associated with the ionisation of its first available hydrogen, and a corresponding anion is formed on loss of this first available hydrogen capable of forming a water-soluble calcium salt, and (ii) following contacting the at least one acid with natural or precipitated calcium carbonate, at least one water-soluble salt, which in the case of a hydrogen-containing salt has a pKa of greater than 7 at 20° C., associated with the ionisation of the first available hydrogen, and the salt anion of which is capable of forming water-insoluble calcium salts, is additionally provided.

“Natural ground calcium carbonate” (GCC) preferably is selected from calcium carbonate containing minerals selected from the group comprising marble, chalk, limestone and mixtures thereof. Natural calcium carbonate may comprise further naturally occurring components such as alumino silicate etc.

In general, the grinding of natural ground calcium carbonate may be a dry or wet grinding step and may be carried out with any conventional grinding device, for example, under conditions such that comminution predominantly results from impacts with a secondary body, i.e. in one or more of: a ball mill, a rod mill, a vibrating mill, a roll crusher, a centrifugal impact mill, a vertical bead mill, an attrition mill, a pin mill, a hammer mill, a pulveriser, a shredder, a de-clumper, a knife cutter, or other such equipment known to the skilled man. In case the calcium carbonate containing mineral material comprises a wet ground calcium carbonate containing mineral material, the grinding step may be performed under conditions such that autogenous grinding takes place and/or by horizontal ball milling, and/or other such processes known to the skilled man. The wet processed ground calcium carbonate containing mineral material thus obtained may be washed and dewatered by well-known processes, e.g. by flocculation, filtration or forced evaporation prior to drying. The subsequent step of drying (if necessary) may be carried out in a single step such as spray drying, or in at least two steps. It is also common that such a mineral material undergoes a beneficiation step (such as a flotation, bleaching or magnetic separation step) to remove impurities.

“Precipitated calcium carbonate” (PCC) in the meaning of the present invention is a synthesized material, generally obtained by precipitation following reaction of carbon dioxide and calcium hydroxide in an aqueous environment or by precipitation of calcium and carbonate ions, for example CaCl₂) and Na₂CO₃, out of solution. Further possible ways of producing PCC are the lime soda process, or the Solvay process in which PCC is a by-product of ammonia production. Precipitated calcium carbonate exists in three primary crystalline forms: calcite, aragonite and vaterite, and there are many different polymorphs (crystal habits) for each of these crystalline forms. Calcite has a trigonal structure with typical crystal habits such as scalenohedral (S-PCC), rhombohedral (R-PCC), hexagonal prismatic, pinacoidal, colloidal (C-PCC), cubic, and prismatic (P-PCC). Aragonite is an orthorhombic structure with typical crystal habits of twinned hexagonal prismatic crystals, as well as a diverse assortment of thin elongated prismatic, curved bladed, steep pyramidal, chisel shaped crystals, branching tree, and coral or worm-like form. Vaterite belongs to the hexagonal crystal system. The obtained PCC slurry can be mechanically dewatered and dried.

According to one embodiment of the present invention, the precipitated calcium carbonate is precipitated calcium carbonate, preferably comprising aragonitic, vateritic or calcitic mineralogical crystal forms or mixtures thereof.

Precipitated calcium carbonate may be ground prior to the treatment with carbon dioxide and at least one H₃O⁺ ion donor by the same means as used for grinding natural calcium carbonate as described above.

According to one embodiment of the present invention, the natural or precipitated calcium carbonate is in form of particles having a weight median particle size d₅₀ of 0.05 to 10.0 μm, preferably 0.2 to 5.0 μm, more preferably 0.4 to 3.0 μm, most preferably 0.6 to 1.2 μm, especially 0.7 μm. According to a further embodiment of the present invention, the natural or precipitated calcium carbonate is in form of particles having a top cut particle size d₉₈ of 0.15 to 55 μm, preferably 1 to 40 μm, more preferably 2 to 25 μm, most preferably 3 to 15 μm, especially 4 μm.

The natural and/or precipitated calcium carbonate may be used dry or suspended in water. Preferably, a corresponding slurry has a content of natural or precipitated calcium carbonate within the range of 1 wt.-% to 90 wt.-%, more preferably 3 wt.-% to 60 wt.-%, even more preferably 5 wt.-% to 40 wt.-%, and most preferably 10 wt.-% to 25 wt.-% based on the weight of the slurry.

The one or more H₃O⁺ ion donor used for the preparation of surface reacted calcium carbonate may be any strong acid, medium-strong acid, or weak acid, or mixtures thereof, generating H₃O⁺ ions under the preparation conditions. According to the present invention, the at least one H₃O⁺ ion donor can also be an acidic salt, generating H₃O⁺ ions under the preparation conditions.

According to one embodiment, the at least one H₃O⁺ ion donor is a strong acid having a pKa of 0 or less at 20° C.

According to another embodiment, the at least one H₃O⁺ ion donor is a medium-strong acid having a pKa value from 0 to 2.5 at 20° C. If the pKa at 20° C. is 0 or less, the acid is preferably selected from sulphuric acid, hydrochloric acid, or mixtures thereof. If the pKa at 20° C. is from 0 to 2.5, the H₃O⁺ ion donor is preferably selected from H₂SO₃, H₃PO₄, oxalic acid, or mixtures thereof. The at least one H₃O⁺ ion donor can also be an acidic salt, for example, HSO₄⁻ or H₂PO₄⁻, being at least partially neutralized by a corresponding cation such as Li⁺, Na⁺ or K⁺, or HPO₄²⁻, being at least partially neutralised by a corresponding cation such as Li⁺, Na⁺, K⁺, Mg²⁺ or Ca²⁺. The at least one H₃O⁺ ion donor can also be a mixture of one or more acids and one or more acidic salts.

According to still another embodiment, the at least one H₃O⁺ ion donor is a weak acid having a pKa value of greater than 2.5 and less than or equal to 7, when measured at 20° C., associated with the ionisation of the first available hydrogen, and having a corresponding anion, which is capable of forming water-soluble calcium salts. Subsequently, at least one water-soluble salt, which in the case of a hydrogen-containing salt has a pKa of greater than 7, when measured at 20° C., associated with the ionisation of the first available hydrogen, and the salt anion of which is capable of forming water-insoluble calcium salts, is additionally provided. According to the preferred embodiment, the weak acid has a pKa value from greater than 2.5 to 5 at 20° C., and more preferably the weak acid is selected from the group consisting of acetic acid, formic acid, propanoic acid, and mixtures thereof. Exemplary cations of said water-soluble salt are selected from the group consisting of potassium, sodium, lithium and mixtures thereof. In a more preferred embodiment, said cation is sodium or potassium. Exemplary anions of said water-soluble salt are selected from the group consisting of phosphate, dihydrogen phosphate, monohydrogen phosphate, oxalate, silicate, mixtures thereof and hydrates thereof. In a more preferred embodiment, said anion is selected from the group consisting of phosphate, dihydrogen phosphate, monohydrogen phosphate, mixtures thereof and hydrates thereof. In a most preferred embodiment, said anion is selected from the group consisting of dihydrogen phosphate, monohydrogen phosphate, mixtures thereof and hydrates thereof. Water-soluble salt addition may be performed dropwise or in one step. In the case of drop wise addition, this addition preferably takes place within a time period of 10 minutes. It is more preferred to add said salt in one step.

According to one embodiment of the present invention, the at least one H₃O⁺ ion donor is selected from the group consisting of hydrochloric acid, sulphuric acid, sulphurous acid, phosphoric acid, citric acid, oxalic acid, acetic acid, formic acid, and mixtures thereof. Preferably the at least one H₃O⁺ ion donor is selected from the group consisting of hydrochloric acid, sulphuric acid, sulphurous acid, phosphoric acid, oxalic acid, H₂PO₄⁻, being at least partially neutralised by a corresponding cation such as Li⁺, Na⁺ or K⁺, HPO₄²⁻, being at least partially neutralised by a corresponding cation such as Li⁺, Na⁺, K⁺, Mg²⁺, or Ca²⁺ and mixtures thereof, more preferably the at least one acid is selected from the group consisting of hydrochloric acid, sulphuric acid, sulphurous acid, phosphoric acid, oxalic acid, or mixtures thereof, and most preferably, the at least one H₃O⁺ ion donor is phosphoric acid.

The one or more H₃O⁺ ion donor can be added to the suspension as a concentrated solution or a more diluted solution. Preferably, the molar ratio of the H₃O⁺ ion donor to the natural or precipitated calcium carbonate is from 0.01 to 4, more preferably from 0.02 to 2, even more preferably 0.05 to 1 and most preferably 0.1 to 0.58.

As an alternative, it is also possible to add the H₃O⁺ ion donor to the water before the natural or precipitated calcium carbonate is suspended.

In a next step, the natural or precipitated calcium carbonate is treated with carbon dioxide. If a strong acid such as sulphuric acid or hydrochloric acid is used for the H₃O⁺ ion donor treatment of the natural or precipitated calcium carbonate, the carbon dioxide is automatically formed. Alternatively or additionally, the carbon dioxide can be supplied from an external source.

H₃O⁺ ion donor treatment and treatment with carbon dioxide can be carried out simultaneously which is the case when a strong or medium-strong acid is used. It is also possible to carry out H₃O⁺ ion donor treatment first, e.g. with a medium strong acid having a pKa in the range of 0 to 2.5 at 20° C., wherein carbon dioxide is formed in situ, and thus, the carbon dioxide treatment will automatically be carried out simultaneously with the H₃O⁺ ion donor treatment, followed by the additional treatment with carbon dioxide supplied from an external source.

In a preferred embodiment, the H₃O⁺ ion donor treatment step and/or the carbon dioxide treatment step are repeated at least once, more preferably several times. According to one embodiment, the at least one H₃O⁺ ion donor is added over a time period of at least about 5 min, preferably at least about 10 min, typically from about 10 to about 20 min, more preferably about 30 min, even more preferably about 45 min, and sometimes about 1 h or more.

Subsequent to the H₃O⁺ ion donor treatment and carbon dioxide treatment, the pH of the aqueous suspension, measured at 20° C., naturally reaches a value of greater than 6.0, preferably greater than 6.5, more preferably greater than 7.0, even more preferably greater than 7.5, thereby preparing the surface-reacted natural or precipitated calcium carbonate as an aqueous suspension having a pH of greater than 6.0, preferably greater than 6.5, more preferably greater than 7.0, even more preferably greater than 7.5.

In a particular preferred embodiment the surface reacted calcium carbonate is a reaction product of natural ground calcium carbonate (GNCC) with carbon dioxide and phosphoric acid, wherein the carbon dioxide is formed in situ by the phosphoric acid treatment.

Further details about the preparation of the surface-reacted natural calcium carbonate are disclosed in WO0039222 A1, WO2004083316 A1, WO2005121257 A2, WO2009074492 A1, EP2264108 A1, EP2264109 A1 and US20040020410 A1, the content of these references herewith being included in the present application.

Similarly, surface-reacted precipitated calcium carbonate is obtained. As can be taken in detail from WO2009074492 A1, surface-reacted precipitated calcium carbonate is obtained by contacting precipitated calcium carbonate with H₃O⁺ ions and with anions being solubilized in an aqueous medium and being capable of forming water-insoluble calcium salts, in an aqueous medium to form a slurry of surface-reacted precipitated calcium carbonate, wherein said surface-reacted precipitated calcium carbonate comprises an insoluble, at least partially crystalline calcium salt of said anion formed on the surface of at least part of the precipitated calcium carbonate.

Said solubilized calcium ions correspond to an excess of solubilized calcium ions relative to the solubilized calcium ions naturally generated on dissolution of precipitated calcium carbonate by H₃O⁺ ions, where said H₃O⁺ ions are provided solely in the form of a counterion to the anion, i.e. via the addition of the anion in the form of an acid or non-calcium acid salt, and in absence of any further calcium ion or calcium ion generating source.

Said excess solubilized calcium ions are preferably provided by the addition of a soluble neutral or acid calcium salt, or by the addition of an acid or a neutral or acid non-calcium salt which generates a soluble neutral or acid calcium salt in situ.

Said H₃O⁺ ions may be provided by the addition of an acid or an acid salt of said anion, or the addition of an acid or an acid salt which simultaneously serves to provide all or part of said excess solubilized calcium ions.

In a further preferred embodiment of the preparation of the surface-reacted natural or precipitated calcium carbonate, the natural or precipitated calcium carbonate is reacted with the one or more H₃O⁺ ion donors and/or the carbon dioxide in the presence of at least one compound selected from the group consisting of silicate, silica, aluminium hydroxide, earth alkali aluminate such as sodium or potassium aluminate, magnesium oxide, or mixtures thereof. Preferably, the at least one silicate is selected from an aluminium silicate, a calcium silicate, or an earth alkali metal silicate. These components can be added to an aqueous suspension comprising the natural or precipitated calcium carbonate before adding the one or more H₃O⁺ ion donors and/or carbon dioxide.

Alternatively, the silicate and/or silica and/or aluminium hydroxide and/or earth alkali aluminate and/or magnesium oxide component(s) can be added to the aqueous suspension of natural or precipitated calcium carbonate while the reaction of natural or precipitated calcium carbonate with the one or more H₃O⁺ ion donors and carbon dioxide has already started. Further details about the preparation of the surface-reacted natural or precipitated calcium carbonate in the presence of at least one silicate and/or silica and/or aluminium hydroxide and/or earth alkali aluminate component(s) are disclosed in WO2004083316 A1.

The surface-reacted calcium carbonate can be kept in suspension, optionally further stabilised by a dispersant. Conventional dispersants known to the skilled person can be used. A preferred dispersant is comprised of polyacrylic acids and/or carboxymethylcelluloses.

Alternatively, the aqueous suspension described above can be dried, thereby obtaining the solid (i.e. dry or containing as little water that it is not in a fluid form) surface-reacted natural or precipitated calcium carbonate in the form of granules or a powder.

In a preferred embodiment, the surface-reacted calcium carbonate has a specific surface area of from 15 m²/g to 200 m²/g, preferably from 20 m²/g to 180 m²/g, more preferably from 25 m²/g to 140 m²/g, even more preferably from 27 m²/g to 120 m²/g, and most preferably from 30 m²/g to 100 m²/g, measured using nitrogen and the BET method. For example, the surface-reacted calcium carbonate has a specific surface area of from 40 m²/g to 100 m²/g, measured using nitrogen and the BET method. The BET specific surface area in the meaning of the present invention is defined as the surface area of the particles divided by the mass of the particles. As used therein the specific surface area is measured by adsorption using the BET isotherm (ISO 9277:2010) and is specified in m²/g.

It is furthermore preferred that the surface-reacted calcium carbonate particles have a volume median particle size d₅₀(vol) from 0.1 to 75 μm, preferably from 0.5 to 50 μm, more preferably from 1 to 40 μm, even more preferably from 1.2 to 30 μm, and most preferably from 1.5 to 15 μm.

It may furthermore be preferred that the surface-reacted calcium carbonate particles have a volume top cut particle size d₉₈(vol) of from 0.2 to 150 μm, preferably from 1 to 100 μm, more preferably from 2 to 80 μm, even more preferably from 2.4 to 60 μm, and most preferably from 3 to 30 μm.

The value dₓ represents the diameter relative to which x % of the particles have diameters less than dₓ. This means that the d₉₈ value is the particle size at which 98% of all particles are smaller. The d₉₈ value is also designated as “top cut”. The dₓ values may be given in volume or weight percent. The d₅₀ (wt) value is thus the weight median particle size, i.e. 50 wt.-% of all grains are smaller than this particle size, and the d₅₀ (vol) value is the volume median particle size, i.e. 50 vol.-% of all grains are smaller than this particle size.

Volume median particle size d₅₀ was evaluated using a Malvern Mastersizer 2000 or 3000 Laser Diffraction System. The d₅₀ or d₉₈ value, measured using a Malvern Mastersizer 2000 or 3000 Laser Diffraction System, indicates a diameter value such that 50% or 98% by volume, respectively, of the particles have a diameter of less than this value. The raw data obtained by the measurement are analysed using the Mie theory, with a particle refractive index of 1.57 and an absorption index of 0.005.

The weight median particle size is determined by the sedimentation method, which is an analysis of sedimentation behaviour in a gravimetric field. The measurement is made with a Sedigraph™ 5100 or 5120, Micromeritics Instrument Corporation. The method and the instrument are known to the skilled person and are commonly used to determine grain size of fillers and pigments. The measurement is carried out in an aqueous solution of 0.1 wt.-% Na₄P₂O₇. The samples were dispersed using a high speed stirrer and sonicated.

The processes and instruments are known to the skilled person and are commonly used to determine grain size of fillers and pigments.

The specific pore volume is measured using a mercury intrusion porosimetry measurement using a Micromeritics Autopore V 9620 mercury porosimeter having a maximum applied pressure of mercury 414 MPa (60 000 psi), equivalent to a Laplace throat diameter of 0.004 μm (— nm). The equilibration time used at each pressure step is 20 seconds. The sample material is sealed in a 5 cm³ chamber powder penetrometer for analysis. The data are corrected for mercury compression, penetrometer expansion and sample material compression using the software Pore-Comp (Gane, P. A. C., Kettle, J. P., Matthews, G. P. and Ridgway, C. J., “Void Space Structure of Compressible Polymer Spheres and Consolidated Calcium Carbonate Paper-Coating Formulations”, Industrial and Engineering Chemistry Research, 35(5), 1996, p 1753-1764.).

The total pore volume seen in the cumulative intrusion data can be separated into two regions with the intrusion data from 214 μm down to about 1-4 μm showing the coarse packing of the sample between any agglomerate structures contributing strongly. Below these diameters lies the fine interparticle packing of the particles themselves. If they also have intraparticle pores, then this region appears bi modal, and by taking the specific pore volume intruded by mercury into pores finer than the modal turning point, i.e. finer than the bi-modal point of inflection, the specific intraparticle pore volume is defined. The sum of these three regions gives the total overall pore volume of the powder, but depends strongly on the original sample compaction/settling of the powder at the coarse pore end of the distribution.

By taking the first derivative of the cumulative intrusion curve the pore size distributions based on equivalent Laplace diameter, inevitably including pore-shielding, are revealed. The differential curves clearly show the coarse agglomerate pore structure region, the interparticle pore region and the intraparticle pore region, if present. Knowing the intraparticle pore diameter range it is possible to subtract the remainder interparticle and interagglomerate pore volume from the total pore volume to deliver the desired pore volume of the internal pores alone in terms of the pore volume per unit mass (specific pore volume). The same principle of subtraction, of course, applies for isolating any of the other pore size regions of interest.

Preferably, the surface-reacted calcium carbonate has an intra-particle intruded specific pore volume in the range from 0.1 to 2.3 cm³/g, more preferably from 0.2 to 2.0 cm³/g, especially preferably from 0.3 to 1.8 cm³/g and most preferably from 0.35 to 1.6 cm³/g, calculated from mercury porosimetry measurement.

The intra-particle pore size of the surface-reacted calcium carbonate preferably is in a range of from 0.004 to 1.6 μm, more preferably in a range of from 0.005 to 1.3 μm, especially preferably from 0.006 to 1.15 μm and most preferably of 0.007 to 1.0 μm, e.g. 0.45 to 0.60 μm determined by mercury porosimetry measurement.

According to one embodiment the surface-reacted calcium carbonate has a volume median particle size d₅₀ from 0.1 to 75 μm, preferably from 0.5 to 50 μm, more preferably from 1 to 40 μm, even more preferably from 1.2 to 30 μm, and most preferably from 1.5 to 15 μm, and

a volume top cut particle size d₉₈ from 0.2 to 150 μm, preferably from 1 to 100 μm, more preferably from 2 to 80 μm, even more preferably from 2.4 to 60 μm, and most preferably from 3 to 30 μm, and

a specific surface area of from 15 m²/g to 200 m²/g, preferably from 20 m²/g to 180 m²/g, more preferably from 25 m²/g to 140 m²/g, even more preferably from 27 m²/g to 120 m²/g, and most preferably from 30 m²/g to 100 m²/g, measured using nitrogen and the BET method, and/or

an intra-particle intruded specific pore volume in the range from 0.1 to 2.3 cm³/g, preferably from 0.2 to 2.0 cm³/g, more preferably from 0.3 to 1.8 cm³/g, and most preferably from 0.35 to 1.6 cm³/g, calculated from mercury porosimetry measurement.

In addition or alternatively, the natural ground calcium carbonate may be selected from the group consisting of marble, chalk, limestone, and mixtures thereof, or

the precipitated calcium carbonate is selected from the group consisting of precipitated calcium carbonates having an aragonitic, vateritic or calcitic crystal form, and mixtures thereof, and/or

the at least one H₃O⁺ ion donor is selected from the group consisting of hydrochloric acid, sulphuric acid, sulphurous acid, phosphoric acid, citric acid, oxalic acid, an acidic salt, acetic acid, formic acid, and mixtures thereof, preferably the at least one H₃O⁺ ion donor is selected from the group consisting of hydrochloric acid, sulphuric acid, sulphurous acid, phosphoric acid, oxalic acid, H₂PO₄⁻, being at least partially neutralised by a cation selected from Li⁺, Na⁺ and/or K⁺, HPO₄²⁻, being at least partially neutralised by a cation selected from Li⁺, Na⁺, K⁺, Mg²⁺, and/or Ca²⁺, and mixtures thereof, more preferably the at least one H₃O⁺ ion donor is selected from the group consisting of hydrochloric acid, sulphuric acid, sulphurous acid, phosphoric acid, oxalic acid, or mixtures thereof, and most preferably, the at least one H₃O⁺ ion donor is phosphoric acid.

According to one embodiment of the present invention, the surface-reacted calcium carbonate comprises an water-insoluble, at least partially crystalline calcium salt of an anion of the at least one acid, which is formed on the surface of the natural ground calcium carbonate or precipitated calcium carbonate. According to one embodiment, the water-insoluble, at least partially crystalline salt of an anion of the at least one acid covers the surface of the natural ground calcium carbonate or precipitated calcium carbonate at least partially, preferably completely. Depending on the employed at least one acid, the anion may be sulphate, sulphite, phosphate, citrate, oxalate, acetate, formiate and/or chloride.

The surface-reacted calcium carbonate may be further treated with a surface-treatment agent or may remain untreated. Suitable surface-treatment agents are, for example, fatty acids, aliphatic carboxylic acids, aliphatic carboxylic esters, mono-substituted succinic anhydrides, mono-substituted succinic acids, or phosphoric acid esters. Suitable surface-treatment agents and methods for preparing surface-treated filler products thereof are, for example, described in EP2159258 A1, EP2390285 A1, EP2390280 A1, WO2014060286 A1 and WO2014128087 A1.

According to one embodiment, the surface-reacted calcium carbonate does not comprise a surface-treatment layer, i.e. an untreated surface-reacted calcium carbonate is used as stabilizing agent.

The Probiotic Composition

According to the present invention, surface-reacted calcium carbonate is used as stabilizing agent for a probiotic composition.

The probiotic composition may be a dry composition or an aqueous suspension. According to a preferred embodiment, the probiotic composition is a dry composition.

The probiotic composition may comprise any probiotic microorganism culture known in the art. According to one embodiment, the probiotic composition comprises a probiotic microorganism culture selected from the group consisting of Bifidobacterium adolescentis, Bifidobacterium lactis, Bifidobacterium infantis, Bifidobacterium longum, Bifidobacterium bifidum, Bifidobacterium breve, Lactobacillus acidophilus, Lactobacillus johnsonii, Lactobacillus casei, Lactobacillus reuteri, Lactobacillus rhamnosus, Lactobacillus plantarum, Lactococcus lactis, Enterococcus faecium, Escherichia coli Nissle 1917, Escherichia coli criodesiccata (083:K24:H31), Saccharomyces boulardii, Saccharomyces cerevisiae, and mixtures thereof. According to one embodiment, the probiotic composition comprises one probiotic microorganism culture. According to another embodiment, the probiotic composition comprises at least two probiotic microorganism cultures, for example, two, three, four, five, six, seven, eight, nine or ten probiotic microorganism cultures.

The probiotic microorganism culture may comprise additional components such as nutrients, bacterial excretions, and other soluble material present in the fermentation cultures of the microorganisms prior to drying. According to one embodiment the probiotic microorganism culture comprises said additional components in an amount of less than 20%, more preferably less than 10% by weight of the probiotic composition.

Methods for growing microorganism cultures are known to the skilled person. For example, the culture may be grown as a pure (single strain) or mixed (multiple strains) culture of the desired microorganisms in a liquid medium, which may be composed of protein or protein fractions, various fermentable carbohydrates, growth stimulants, inorganic salts, buffers etc, in sterile whole milk, skim milk, whey, or other natural substrates, or combinations thereof. After inoculation, the culture is allowed to develop under generally optimized incubation conditions of time and temperature. Depending on the microorganism being grown, the incubation times may range from periods of 4 to 48 hours, and the temperatures may vary from 15° C. to 50° C. It may also be desirable to control pH and dissolved oxygen. After satisfactory growth has been attained, the culture in its growth medium is typically cooled to between 0° C. to 15° C. After fermentation, the liquid medium may be removed from the bacterial culture by any suitable method known in the art such as centrifugation, ultrafiltration, or sedimentation.

According to one embodiment, the probiotic composition comprises a probiotic microorganism culture in an amount of at least 50 wt.-%, based on the total weight of the probiotic composition, more preferably in an amount of at least 75 wt.-%, even more preferably in an amount of at least 90 wt.-%, and most preferably in an amount of at least 95 wt.-%. According to one embodiment the probiotic composition consists of the probiotic microorganism culture.

The probiotic composition may comprise further additives to enhance its performance, for example, vitamins, enzymes, plasticizers, coloring agents, flavoring agents, sweeteners, anti-oxidants, buffering agents, slip aids, or mixtures thereof.

Examples of suitable vitamins are vitamin A, vitamin B1, vitamin B2, vitamin B6, vitamin B12, niacin, folic acid, biotin, vitamin C, vitamin D, vitamin E, vitamin K, or mixtures thereof. An example of a suitable enzyme is a proteolytic enzyme such as pancreatin. Examples of suitable coloring agents are food colorants, riboflavin, β-carotene, or natural coloring agents, preferably fruit, vegetable, and/or plant extracts such as grape, black currant, aronia, carrot, beetroot, red cabbage, and hibiscus. The flavoring agents may be selected from any suitable natural or synthetic flavor agent, for example, passion fruit flavors, mango flavors, pineapple flavors, cupuacu flavors, guava flavors, cocoa flavors, papaya flavors, peach flavors, apricot flavors, apple flavors, citrus flavors, grape flavors, raspberry flavors, cranberry flavors, cherry flavors, or grapefruit flavors.

Examples of suitable sweeteners are carbohydrate sweeteners, preferably monosaccharides and/or disaccharides, more preferably sucrose, fructose, glucose, maltose, and mixtures thereof; saccharin, cyclamates, L-aspartyl-L-phenylalanine lower alkyl ester sweeteners (e.g., aspartame); thaumatin; dihydrochalcones; cyclamates; steviosides; glycyrrhizins, synthetic alkoxy aromatics; sucralose; suosan; miraculin; monellin; sorbitol, xylitol; talin; cyclohexylsulfamates; substituted imidazolines; synthetic sulfamic acids such as acesulfame, acesulfame K and n-substituted sulfamic acids; oximes such as perilartine; peptides such as aspartyl malonates and succanilic acids; dipeptides; amino acid based sweeteners such as gem-diaminoalkanes, meta-aminobenzoic acid, L-aminodicarboxylic acid alkanes, and amides of certain alpha-aminodicarboxylic acids and gem-diamines; and 3-hydroxy-4-alkyloxyphenyl aliphatic carboxylates or heterocyclic aromatic carboxylates; erythritol; and mixtures thereof.

Examples of suitable anti-oxidants include tocopherols (e.g., vitamin E), ascorbic acid (e.g., vitamin C), vitamin A (e.g., beta-carotene), grape seed extract, selenium, and coenzyme Q10, butylated hydroxytoluene (BHT), butylated hydroxyanisole (BHA), propyl gallate, and mixtures thereof.

Other non-limiting examples of optional components useful in the compositions of the present invention include diclofenac, naproxen, aspirin, indomethacin, omeprazole, cardiac glycosides, electrolyte preparations with sodium, potassium, or magnesium salts as well as calcium and iron preparations, bisacodyl preparations, valproic acid, 5-ASA, steroids such as hydrocortisone, budesonide, laxatives, octreotide, cisapride, anticholinergies, calcium channel blockers, 5HT3-antagonists such as ondansetron and peptides such as insulin, solid lubricants such as stearic acid and magnesium stearate; calcium sulfate; vegetable oils such as peanut oil, cottonseed oil, sesame oil, olive oil, corn oil and oil of theobroma; emulsifiers such as TWEENS; wetting agents such as sodium lauryl sulfate; tabletting agents such as binders, antioxidants; or preservatives.

According to one embodiment, the probiotic composition does not contain sugars and/or polysaccharides, preferably the probiotic composition does not contain maltodextrin.

The inventors of the present invention surprisingly found that the addition of surface-reacted calcium carbonate to a probiotic composition can reduce the inactivation of the probiotic microorganism culture during drying and storage of the probiotic composition. In particular, it was surprisingly found that surface-reacted calcium carbonate as defined herein can minimize cell death during drying of the probiotic composition. Without being bound to any theory it is believed that the pH of the surface-reacted calcium carbonate, which is about 7 in an aqueous solution, and its low water activity have a beneficial effect on the stability of the probiotic microorganism culture.

According to one embodiment surface-reacted calcium carbonate may be used as a drying stabilizer and/or shelf live preservative. Thus, use of surface-reacted calcium carbonate as drying stabilizer and/or shelf live preservative for a probiotic composition is provided, wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source.

According to one embodiment, the weight ratio of probiotic microorganism culture:surface-reacted calcium carbonate is from 5:95 to 40:60, preferably from 10:90 to 35:65, more preferably from 15:85 to 30:70, and most preferably from 20:80 to 25:75.

The probiotic composition may have a concentration of viable probiotic microorganism culture of at least 10⁶ CFU/g. Methods for determining the viability of the microorganism culture are known in the art. For example, the viability of probiotic microorganism culture may be determined by plating the probiotic microorganism culture on a suitable medium, e.g. solidified agar on a standard sized Petri dish, and determine the numbers of colonies formed. The unit CFU (colony-forming unit) is used to quantify the amount of viable probiotic microorganisms in the probiotic composition.

According to one embodiment, the probiotic composition has a viable probiotic microorganism culture concentration from 10⁶ CFU/g to 10¹⁴ CFU/g, preferably from 10⁷ CFU/g to 10¹³ CFU/g, and most preferably from 10⁸ CFU/g to 10¹² CFU/g. According to a further embodiment, the dry probiotic composition has a viable probiotic microorganism culture concentration from 10⁶ CFU/g to 10¹⁴ CFU/g, preferably from 10⁷ CFU/g to 10¹³ CFU/g, and most preferably from 10⁸ CFU/g to 10¹² CFU/g.

The concentration of viable probiotic microorganism culture is measured directly after drying the probiotic composition or after a certain storage time. According to one embodiment the probiotic composition has a viable probiotic microorganism culture concentration from 10⁶ CFU/g to 10¹⁴ CFU/g, preferably from 10⁷ CFU/g to 10¹³ CFU/g, and most preferably from 10⁸ CFU/g to 10¹² CFU/g, after drying the probiotic composition, preferably after spray drying the probiotic composition. In addition or alternatively, the probiotic composition may have a viable probiotic microorganism culture concentration from 10⁶ CFU/g to 10¹⁴ CFU/g, preferably from 10⁷ CFU/g to 10¹³ CFU/g, and most preferably from 10⁸ CFU/g to 10¹² CFU/g, after storing the dry probiotic composition for 2 weeks at 30° C. and a humidity of 35%.

The inventors of the present invention surprisingly found that surface-reacted calcium carbonate as defined herein may provide a better stabilizing performance than conventionally used stabilizing agents such as maltodextrin.

According to one embodiment, the concentration of viable probiotic microorganism culture after drying of the probiotic composition is increased by at least 5%, preferably by at least 10%, more preferably by at least 15%, and most preferably by at least 20%, compared to a probiotic composition comprising maltodextrin as stabilizing agent. According to one embodiment, the drying is spray drying.

According to a further aspect of the present invention, a method for stabilizing a probiotic microorganism culture is provided, comprising the step of mixing a probiotic microorganism culture with a surface-reacted calcium carbonate in an aqueous medium, wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source, and drying the obtained mixture.

The drying may be carried out by any suitable method known to the skilled person. According to one embodiment, the drying is carried out by spray drying, freeze drying, spray freeze drying, flash drying, fluidized bed drying, jet drying, vacuum drying, or a combination thereof, preferably by spray drying, freeze drying, spray freeze drying, or a combination thereof, and most preferably spray drying.

Spray drying is a process well-known industrial and economic process in the art, in which particles are formed at the same time as they are dried. Dry granulated powders may be produced from a wet product such as a slurry or solution, by atomizing the wet product via a rotary wheel or nozzle at high velocity and directing the spray of droplets into a flow of hot air e.g. 130 to 210° C. The atomized droplets have a very large surface area in the form of millions of micrometer-sized droplets (e.g. 10 to 200 μm), which results in a very short drying time when exposed to hot air in a drying chamber.

During freeze drying, the mixture is first frozen to below the critical temperature of the formulation, and then dried by sublimation under high vacuum in two phases: primary drying, during which unbound water is removed and secondary drying, during which the bound water is removed.

Spray freeze drying is essentially a combination of spray drying and freeze drying, wherein a wet product is sprayed by an atomization nozzle into a cold vapor phase of a cryogenic liquid, such a liquid nitrogen, so the droplets may start freezing during their passage through the cold vapor phase, and completely freeze upon contact with the cryogenic liquid phase. The frozen droplets are then dried by freeze drying.

According to a preferred embodiment, the probiotic composition is dried by spray drying. Suitable equipment and methods are known to the skilled person. For example, the spray drying step may be carried out with a fluidized bed dryer. The skilled person will adapt the drying temperature and drying time accordingly. For example, the spray drying may be carried out at an inlet temperature from 130 to 210° C. and an outlet temperature from 50 to 130° C. According to one embodiment the spray drying is carried out at an inlet temperature from 140 to 200° C., preferably from 150 to 190° C., more preferably from 160 to 180, and most preferably at about 170° C., and/or at an outlet temperature from 60 to 120° C., preferably from 65 to 110° C., more preferably from 70 to 100° C., even more preferably from 75 to 90° C., and most preferably at about 80° C.

According to a further aspect of the present invention, a process for preparing a dry stabilized probiotic composition is provided, wherein the process comprises the steps of:

a) providing an aqueous probiotic composition comprising at least 75 wt.-% of a probiotic microorganism culture, based on the total weight of the probiotic composition,

b) providing an aqueous suspension comprising 10 to 30 wt.-%, based on the total weight of the aqueous suspension, of surface-reacted calcium carbonate, wherein the surface-reacted calcium carbonate is a reaction product of natural ground calcium carbonate or precipitated calcium carbonate with carbon dioxide and one or more H₃O⁺ ion donors selected from the group consisting of hydrochloric acid, sulphuric acid, sulphurous acid, phosphoric acid, oxalic acid, or mixtures thereof, wherein the carbon dioxide is formed in situ by the H₃O⁺ ion donors treatment and/or is supplied from an external source,

wherein the surface-reacted calcium carbonate has a volume median particle size d₅₀ from 0.1 to 75 μm, and a volume top cut particle size d₉₈ from 0.2 to 150 μm, a specific surface area of from 15 m²/g to 200 m²/g, measured using nitrogen and the BET method, and

wherein the weight ratio of probiotic microorganism culture:surface-reacted calcium carbonate is from 5:95 to 40:60,

c) mixing the probiotic composition of step a) and the surface-reacted calcium carbonate of step b), and

d) spray drying the mixture obtained in step c), at an inlet temperature from 130 to 210° C. and an outlet temperature from 50 to 130° C.

According to a further aspect of the present invention, a dry stabilized probiotic composition obtainable by the process according to the present invention is provided. Thus, a dry stabilized probiotic composition is provided obtained by a process comprising the steps of:

a) providing an aqueous probiotic composition comprising at least 75 wt.-% of a probiotic microorganism culture, based on the total weight of the probiotic composition,

b) providing an aqueous suspension comprising 10 to 30 wt.-%, based on the total weight of the aqueous suspension, of surface-reacted calcium carbonate, wherein the surface-reacted calcium carbonate is a reaction product of natural ground calcium carbonate or precipitated calcium carbonate with carbon dioxide and one or more H₃O⁺ ion donors selected from the group consisting of hydrochloric acid, sulphuric acid, sulphurous acid, phosphoric acid, oxalic acid, or mixtures thereof, wherein the carbon dioxide is formed in situ by the H₃O⁺ ion donors treatment and/or is supplied from an external source,

wherein the surface-reacted calcium carbonate has a volume median particle size d₅₀ from 0.1 to 75 μm, and a volume top cut particle size d₉₈ from 0.2 to 150 μm, a specific surface area of from 15 m²/g to 200 m²/g, measured using nitrogen and the BET method, and

wherein the weight ratio of probiotic microorganism culture:surface-reacted calcium carbonate is from 5:95 to 40:60,

c) mixing the probiotic composition of step a) and the surface-reacted calcium carbonate of step b), and

d) spray drying the mixture obtained in step c), at an inlet temperature from 130 to 210° C. and an outlet temperature from 50 to 130° C.

The dry stabilized probiotic composition may be in form of a particulate material, for example, in form of granules, powders, grains, tablets, or crumbles. Subsequently, the dry stabilized probiotic composition may be shaped into any other suitable form. For example, said composition may be subjected to a grinding process or a compacting process.

The probiotic composition may be employed in a wide range of applications, and the skilled person will adapt the composition and form of the probiotic composition for the desired application. According to one embodiment, the probiotic composition is a pharmaceutical probiotic composition, a nutritional probiotic composition, or a cosmetic probiotic composition. The probiotic composition may be included in various products. According to one embodiment, the probiotic composition is comprised by a tablet, a capsule, a chewable tablet, a chewable gum, a chewable pastille, a lozenge, a powder, a granulate, a pellet, a paste, a cream, a food, a feed, or a beverage.

According to a further aspect of the present invention, a product comprising the dry stabilized probiotic composition according to the present invention is provided, wherein the product is a tablet, a capsule, a chewable tablet, a chewable gum, a chewable pastille, a lozenge, a powder, a granulate, a pellet, a paste, a cream, a food, a feed, or a beverage.

A “food” according to the present invention is any product that is intended for human consumption, while a “feed” refers to a product that is intended for animal consumption. Examples of food products are yogurt, fermented vegetables, milk powder, or vitamin/mineral complexes. Examples of feed products are pet milk as well as dry or wet pet food. Examples of beverages are milks, fermented milks, lactic acid bacteria beverages, fermented vegetable beverages, fermented fruit beverages, plant milk beverages, or fermented plant milk beverages. Non-limiting examples of plant milks that can be used for plant milk beverages or fermented plant milk beverages are oat milk, rice milk, soy milk, almond milk, hemp milk, coconut milk, lupine milk, pea milk, barley milk, or hazelnut milk. Examples of cosmetic probiotic compositions are probiotic moisturizer, probiotic cream, probiotic gel, probiotic serum, probiotic shower gel, probiotic soap, probiotic shampoo, probiotic face wash, probiotic skin mask, or probiotic skin spray.

According to still a further aspect of the present invention, use of a dry stabilized probiotic composition according to the present invention in pharmaceutical, nutritional or cosmetic applications is provided.

According to one embodiment use of surface-reacted calcium carbonate as stabilizing agent for a probiotic composition is provided, wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source,

wherein the surface-reacted calcium carbonate has a volume median particle size d₅₀ from 1.2 to 30 μm, preferably from 1.5 to 15 μm, and volume top cut particle size d₉₈ from 2.4 to 60 μm, preferably from 3 to 30 μm, and a specific surface area from 27 m²/g to 120 m²/g, preferably from 30 m²/g to 100 m²/g, measured using nitrogen and the BET method, and an intra-particle intruded specific pore volume in the range from 0.35 to 1.6 cm³/g, calculated from mercury porosimetry measurement,

the probiotic composition comprises a probiotic microorganism culture selected from the group consisting of Bifidobacterium adolescentis, Bifidobacterium lactis, Bifidobacterium infantis, Bifidobacterium longum, Bifidobacterium bifidum, Bifidobacterium breve, Lactobacillus acidophilus, Lactobacillus johnsonii, Lactobacillus casei, Lactobacillus reuteri, Lactobacillus rhamnosus, Lactobacillus plantarum, Lactococcus lactis, Enterococcus faecium, Escherichia coli Nissle 1917, Escherichia coli criodesiccata (083:K24:H31), Saccharomyces boulardii, Saccharomyces cerevisiae, and mixtures thereof, and the weight ratio of probiotic microorganism culture:surface-reacted calcium carbonate is from 5:95 to 40:60, preferably from 10:90 to 35:65, more preferably from 15:85 to 30:70, and most preferably from 20:80 to 25:75.

According to a further embodiment of the present invention, a process for preparing a dry stabilized probiotic composition is provided, wherein the process comprises the steps of:

a) providing an aqueous probiotic composition comprising at least 75 wt.-% of a probiotic microorganism culture, based on the total weight of the probiotic composition,

b) providing an aqueous suspension comprising 10 to 30 wt.-%, based on the total weight of the aqueous suspension, of surface-reacted calcium carbonate, wherein the surface-reacted calcium carbonate is a reaction product of natural ground calcium carbonate or precipitated calcium carbonate with carbon dioxide and one or more H₃O⁺ ion donors selected from the group consisting of phosphoric acid, wherein the carbon dioxide is formed in situ by the H₃O⁺ ion donors treatment,

wherein the surface-reacted calcium carbonate has a volume median particle size d₅₀ from 1.2 to 30 μm, preferably from 1.5 to 15 μm, and volume top cut particle size d₉₈ from 2.4 to 60 μm, preferably from 3 to 30 μm, and a specific surface area from 27 m²/g to 120 m²/g, preferably from 30 m²/g to 100 m²/g, measured using nitrogen and the BET method, and an intra-particle intruded specific pore volume in the range from 0.35 to 1.6 cm³/g, calculated from mercury porosimetry measurement, and wherein the weight ratio of probiotic microorganism culture:surface-reacted calcium carbonate is from 5:95 to 40:60,

c) mixing the probiotic composition of step a) and the surface-reacted calcium carbonate of step b), and

d) spray drying the mixture obtained in step c), at an inlet temperature from 160 to 180° C. and an outlet temperature from 70 to 90° C.

The scope and interest of the present invention will be better understood based on the following figures and examples which are intended to illustrate certain embodiments of the present invention and are non-limitative.

### EXAMPLES

### 1. Methods

In the following, measurement methods implemented in the examples are described.

### Particle Size Distribution

Volume determined median particle size d₅₀ (vol) and the volume determined top cut particle size d₉₈(vol) was evaluated using a Malvern Mastersizer 3000 Laser Diffraction System (Malvern Instruments Plc., Great Britain). The d₅₀ (vol) or d₉₈(vol) value indicates a diameter value such that 50% or 98% by volume, respectively, of the particles have a diameter of less than this value. The raw data obtained by the measurement was analyzed using the Mie theory, with a particle refractive index of 1.57 and an absorption index of 0.005. The methods and instruments are known to the skilled person and are commonly used to determine particle size distributions of fillers and pigments. The measurement was carried out in an aqueous solution of 0.1 wt.-% Na₄P₂O₇. The samples were dispersed using a high-speed stirrer and supersonicated.

The weight median particle size d₅₀ (wt) is determined by the sedimentation method, which is an analysis of sedimentation behaviour in a gravimetric field. The measurement is made with a Sedigraph™ 5120, Micromeritics Instrument Corporation. The method and the instrument are known to the skilled person and are commonly used to determine grain size of fillers and pigments. The measurement is carried out in an aqueous solution of 0.1 wt % Na₄P₂O₇. The samples were dispersed using a high speed stirrer and supersonicated.

The processes and instruments are known to the skilled person and are commonly used to determine grain size of fillers and pigments.

### Specific Surface Area (SSA)

The specific surface area was measured via the BET method according to ISO 9277:2010 using nitrogen, following conditioning of the sample by heating at 250° C. for a period of 30 minutes. Prior to such measurements, the sample was filtered within a Büchner funnel, rinsed with deionised water and dried at 110° C. in an oven for at least 12 hours.

Intra-Particle Intruded Specific Pore Volume (in cm³/q)

The specific pore volume was measured using a mercury intrusion porosimetry measurement using a Micromeritics Autopore V 9620 mercury porosimeter having a maximum applied pressure of mercury 414 MPa (60 000 psi), equivalent to a Laplace throat diameter of 0.004 μm (— nm). The equilibration time used at each pressure step was 20 seconds. The sample material was sealed in a 5 cm³ chamber powder penetrometer for analysis. The data were corrected for mercury compression, penetrometer expansion and sample material compression using the software Pore-Comp (Gane, P. A. C., Kettle, J. P., Matthews, G. P. and Ridgway, C. J., “Void Space Structure of Compressible Polymer Spheres and Consolidated Calcium Carbonate Paper-Coating Formulations”, Industrial and Engineering Chemistry Research, 35(5), 1996, p 1753-1764.).

The total pore volume seen in the cumulative intrusion data can be separated into two regions with the intrusion data from 214 μm down to about 1-4 μm showing the coarse packing of the sample between any agglomerate structures contributing strongly. Below these diameters lies the fine inter-particle packing of the particles themselves. If they also have intra-particle pores, then this region appears bi-modal, and by taking the specific pore volume intruded by mercury into pores finer than the modal turning point, i.e. finer than the bi-modal point of inflection, the specific intra-particle pore volume is defined. The sum of these three regions gives the total overall pore volume of the powder, but depends strongly on the original sample compaction/settling of the powder at the coarse pore end of the distribution.

By taking the first derivative of the cumulative intrusion curve the pore size distributions based on equivalent Laplace diameter, inevitably including pore-shielding, are revealed. The differential curves clearly show the coarse agglomerate pore structure region, the inter-particle pore region and the intra-particle pore region, if present. Knowing the intra-particle pore diameter range it is possible to subtract the remainder inter-particle and inter-agglomerate pore volume from the total pore volume to deliver the desired pore volume of the internal pores alone in terms of the pore volume per unit mass (specific pore volume). The same principle of subtraction, of course, applies for isolating any of the other pore size regions of interest.

### 2. Materials

Stabilizer

SRCC: Surface-reacted calcium carbonate (d₅₀ (vol)=6.6 μm, d₉₈ (vol)=13.7 μm, SSA=59.9 m²/g). The intra-particle intruded specific pore volume is 0.939 cm³/g (for the pore diameter range of 0.004 to 0.51 μm).

SRCC was obtained by preparing 350 litres of an aqueous suspension of ground calcium carbonate in a mixing vessel by adjusting the solids content of a ground limestone calcium carbonate from Omya SAS, Orgon having a weight based median particle size d₅₀ (wt) of 1.3 μm, as determined by sedimentation, such that a solids content of 10 wt.-%, based on the total weight of the aqueous suspension, is obtained.

Whilst mixing the slurry at a speed of 6.2 m/s, 11.2 kg phosphoric acid was added in form of an aqueous solution containing 30 wt.-% phosphoric acid to said suspension over a period of 20 minutes at a temperature of 70° C. After the addition of the acid, the slurry was stirred for additional 5 minutes, before removing it from the vessel and drying using a jet-dryer.

Maltodextrin (comparative stabilizer).

### Probiotic Microorganism

Lactobacillus plantarum WCFS1.

### 3. Example 1

### 3.1. Fermentation and Harvesting of Probiotic

Firstly, a pre-inoculum was made from Lactobacillus plantarum strain in 5 mL of MRS-B culture medium. This was incubated for approximately 10 hours at 37° C. Afterwards, 1% of the pre-inoculum was used to inoculate the actual fermentation. The fermentation medium used was again MRS-B, which was then incubated over night (˜16-17 hours) at a temperature of 37° C. After the fermentation, a sample was taken for CFU determination.

For harvesting, the finished fermentation was centrifuged for 15 minutes at 5000 rpm (4° C.). The resulting pellet was resuspended in 100 mL of PBS (phosphate-buffered saline) buffer and subdivided in 2 equally sized portions. The concentration of probiotic microorganisms in said suspensions was 4.2×10¹⁰ CFU/g. Each of these portions was mixed with 450 g of stabilizer solution containing 20% (w/w) of stabilizer, resulting in 2 solutions of −500 g. One of the stabilizer solutions contained maltodextrin while the other contained the inventive stabilizing agent. Note that both stabilizer solutions were sterilized (15 minutes at 121° C.) before mixing with the resuspended pellet portions.

### 3.2 Spray Drying and Shelf Life Testing

Before spray drying, a sample of each stabilizer solution was taken for CFU determination before drying. The CFU analysis was carried out according to ISO 11133-1:2009.

For the spray drying trial, a Büchi benchtop spray drying system was used. An inlet temperature of 170° C. and an outlet temperature of 80° C. as used to spray dry all material. Again, the resulting powder was sampled for CFU analysis for yield determination after drying.

The remaining powder was used for shelf life analysis. For each stabilizer powder, 0.5-1 g was dosed in reactor tubes with filter caps. These tubes were consequently incubated horizontally (for a larger air contact area) at 30° C. with a humidity of 35% for 2 weeks. After this incubation period, samples were again taken for CFU analysis.

### 3.3 Results

The results for the CFU measurements at different steps in the benchmark study are shown in Table 1. It can be seen that the concentration of viable probiotic microorganisms (CFU/g) of spray dried powder decreases from before spray drying all the way to the end of the shelf life study. The amount after fermentation appears to be slightly lower than the amount before spray drying, but this is simply caused by the approximation of the dry matter used for the back calculation of this value. Additionally, the standard deviation for each of the measurements is relatively high (as is usually for CFU determinations). These numbers for the standard deviations are shown in Table 2.

With regards to product properties, the maltodextrin solution before spray drying was clear, while the suspension of the inventive stabilizing agent was turbid (milky). The spray dried maltodextrin powder was more difficult to resuspend then the mineral stabilizer powder, though the latter was prone settling. The maltodextrin powder was also much denser with a bulk density of around 470 g/L compared to the approximate 200 g/L for the mineral stabilizer powder.

TABLE 1
CFU/grams of powder for each step in the benchmark
study. The concentration after fermentation was
back calculated based on approximate dry matter.

| | Maltodextrin | |
|---|---|---|
| | (comparative) | SRCC |
| | (CFU/g) | (CFU/g) |
| After fermentation (back calculated) | 4.2E+10 | |
| Before spray drying | 6.19E+10 | 5.91E+10 |
| After spray drying | 1.09E+10 | 1.45E+10 |
| After shelf life testing | 9.16E+07 | 1.95E+08 |

TABLE 2
Standard deviation for CFU determination
for each step in the benchmark study.

| | Maltodextrin | |
|---|---|---|
| | (comparative) | SRCC |
| | (CFU/g) | (CFU/g) |
| After fermentation (back calculated) | 1.2E+10 | |
| Before spray drying | 2.64E+10 | 2.51E+10 |
| After spray drying | 2.73E+9 | 6.42E+9 |
| After shelf life testing | 4.35E+07 | 1.42E+08 |

The results compiled in Tables 1 and 2 show that the concentration of viable probiotic microorganisms is significantly higher in the inventive sample after spray drying and the inventive sample exhibits a significantly increased shelf life.

### 4. Example 2

### 4.1. Fermentation and Harvesting of Probiotic

Firstly, a pre-inoculum was made from Lactobacillus plantarum strain in 5 mL of MRS-B culture medium. This was incubated for approximately 10 hours at 37° C. Afterwards, 1% of the pre-inoculum was used to inoculate the actual fermentation. The fermentation medium used was again MRS-B, which was then incubated over night (˜16-17 hours) at a temperature of 37° C. After the fermentation, a sample was taken for CFU determination.

For harvesting, the finished fermentation was centrifuged for 15 minutes at 5000 rpm (4° C.). The resulting pellet was resuspended in 250 mL of PBS (phosphate-buffered saline) buffer and subdivided in five equally sized portions. Three of these portions was mixed with 450 g of a stabilizer solution containing (based on the total weight of the final mixture):
Stabilizer solution 1: only PBS (control buffer solution)
Stabilizer solution 2: 5 wt.-% maltodextrin
Stabilizer solution 3: 5 wt.-% SRCC

This resulted in three sample solutions of 500 g. The stabilizer solutions were sterilized (15 minutes at 121° C.) before mixing with the resuspended pellet portions. The composition of the sample solutions is indicated in Table 3 below.

TABLE 3
Composition of sample solutions
produced according to Example 2.

| Sample | Amount of PBS | Stabilizing | Biomass | Total solids |
|---|---|---|---|---|
| solution | [g] | agent | [g] | [wt.-%] |
| 1 | 4.95 | — | ~5 | 1.99 |
| 2 | 0.494 | 5 g Maltodextrin | ~5 | 2.10 |
| 3 | 0.494 | 5 g SRCC | ~5 | 2.10 |

### 4.2. Spray Drying

Before spray drying, a sample of each sample solution was taken for CFU determination as reference. A Büchi benchtop dryer was used for spray drying.

For phase 1, an inlet temperature of 200° C. and an outlet temperature of 100° C. was used to spray dry all sample solutions. Since the main aim of phase 1 was to obtain observable yield differences between the different sample solutions, the inlet and outlet temperature were set relatively high to ensure more inactivation and consequently more contrast between the samples.

During phase 2 the drying conditions were, 180 and 80° C. for inlet and outlet temperature respectively. The main aim of phase 2 was to define effect of stabilizers and formulations on gut and shelf-life survival. Consequently, relatively mild spray drying conditions were chosen to maximize starting CFU's for consequent testing.

### 4.3. Formulations (Phase 2)

Three different probiotic formulations were prepared to further asses possible applications of the inventive stabilizing agents, using the spray dried sample solutions 2 and 3 containing the stabilizing agent maltodextrin and SRCC, respectively, and subjected to digestion and/or shelf life experiments. For the different formulations, the powders, which were obtained by spray drying the corresponding sample solutions according to the phase 2 conditions, were mixed with specific bulk components indicated below in a weight ratio of 1:1.

### Milk Powder for Liquid Ingestion

Spray dried powders were mixed in a 1:1 weight ratio with standard skim milk powder. Part of this dry blend was used for shelf life analysis (dry) while the other part was solubilized for the digestion study.

### Tablet for Ingestion

Spray dried powders were mixed in a 1:1 weight ratio with lactose powder. This powder blend was then manually pressed into tablets of −1 gram. These tablets were subjected to the digestion study and shelf life experiments.

### Cream for Skin Application

A base cream was prepared by mixing oleylalcohol and hexadecanol in a 1:1 weight ratio, creating a Vaseline-like cream. Cream and spray dried powder was then mixed in a 1:1 weight ratio, after which it was subjected to shelf life analysis.

### 4.4. Digestion Study and Shelf Life Experiments

### In Vitro-Digestion Testing

An in-vitro digestion model was used to evaluate the gut survival of L. Plantarum WCFS1 using different stabilizers and formulations. This model has been validated to show comparable strain-specific GI persistence to in-vivo methods (Van Bokhorst-van de Veen et al., 2012).

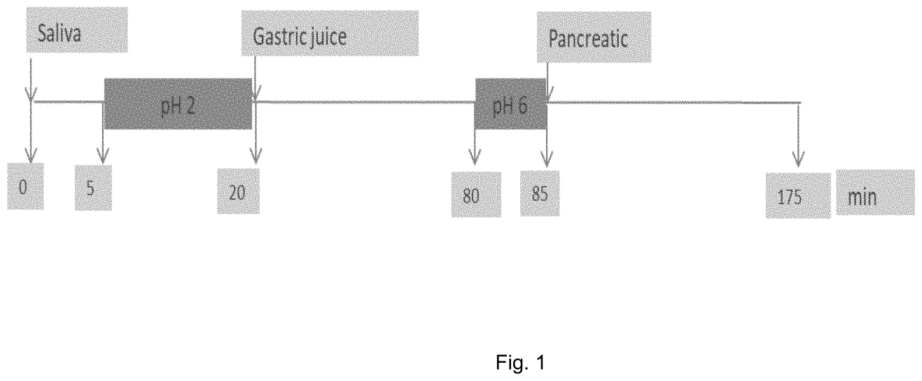

The digestion study was done in an in-vitro setup, using a glass sample holder inside a shaken water bath to simulate intestinal mixing. Initially, 3 grams of formulation (milk powder dry blend or tablets) were added to 27 g of sterile water in a sample holder (resulting in a 10 wt.-% solution). This was done to simulate practical intake of a typical milk powder solution and tablets with water respectively. The four resulting formulations were subjected to the in-vitro digestion scheme, as presented in FIG. 1, in which the time of addition of different digestive juices and setting of pH is indicated in minutes. The in-vitro digestion scheme included the following steps:
0 minutes: Addition of 2 mL saliva buffer, containing α-amylase
5 minutes: Adjust pH to 2 using 0.5 M HCl solution over 15 minutes.
20 minutes: Addition of 8 mL gastric buffer, containing lipase and pepsin.
80 minutes: Take 0.3 mL sample (after gastric phase) and start adjusting pH to 6 using 1 M NaOH solution over 5 minutes.
85 minutes: Addition of 10 mL pancreatic buffer, containing pancreatin and bile.
175 minutes: Take 0.3 mL sample (after pancreatic phase) and finalize experiment.
   The samples taken were then used for CFU analysis.

### Shelf Life Testing

For shelf life experiments, the same formulations as used for the digestion study were evaluated, as well as the cream for skin application. For each formulation, 0.5-1 g was dosed in reactor tubes with filter caps. These tubes were incubated horizontally (for a larger air contact area) at 30° C. with a humidity of 35% for 2 weeks. After this incubation period, samples were taken for CFU analysis.

### 4.5. Results

### Spray Drying

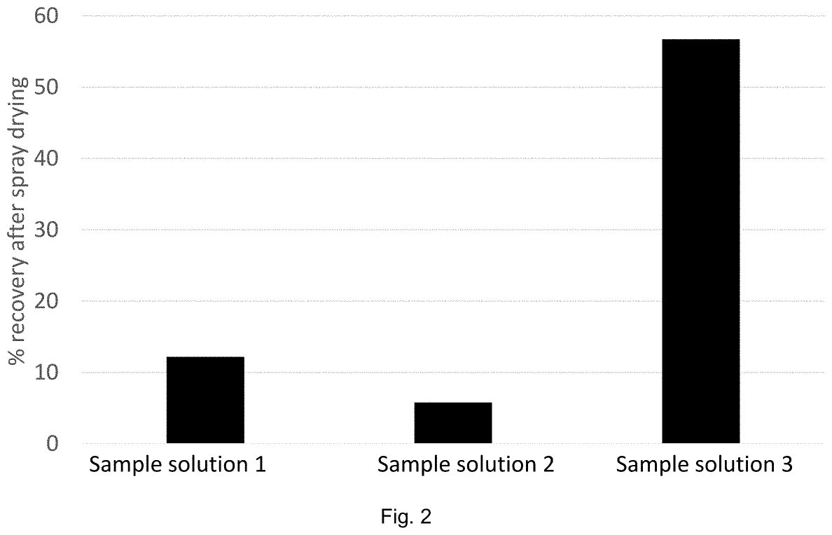

After spray drying a CFU analysis of the buffer solution (control sample) and the stabilized samples was carried out according to ISO 1133-1:2009. The results are compiled in FIG. 2. Comparison in terms of recovery % is generally applied in the literature (Perdana et al. 2013). As can be seen from FIG. 2, the CFU values for the inventive sample comprising SRCC are consistently higher than for the comparative sample comprising maltodextrin. The yield for the maltodextrin sample was in line with literature values of 5-10% (Siemons et al. 2021), while the yield for the control sample with buffer was relatively high compared to maltodextrin.

Furthermore, there was less material on the wall of the Büchi for the inventive samples comprising SRCC compared to the sample containing maltodextrin. The spray dried maltodextrin sample also had more powder lumps which were less easily dispersible into lose powder.

### In-Vitro Digestion Testing

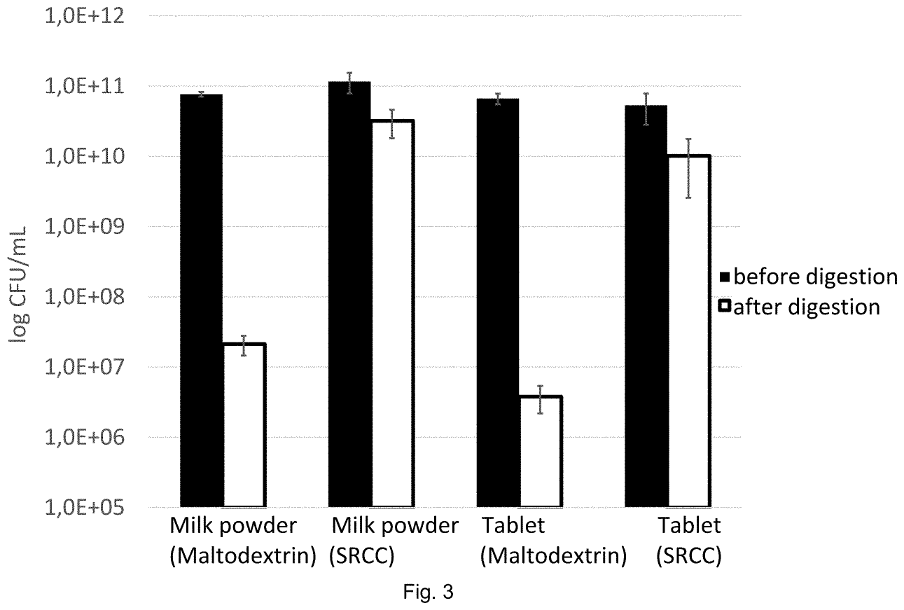

A CFU analysis of the stabilized samples was carried out according to ISO 1133-1:2009, before and after the in-vitro digestion test. The results are shown in FIG. 3. In this case, the difference in performance for the samples containing the inventive stabilizing agent SRCC and the comparative samples containing maltodextrin is striking. The inventive samples with SRCC maintained 10% viability, i.e., a log 1 reduction after digestion. In contrast, the comparative samples with maltodextrin were more sensitive, with only 0.01% viability remaining, i.e., a log 4 reduction after digestion. The inventive stabilizing agent SRCC outperformed maltodextrin in both formulations, i.e., milk powder as well as lactose tablets.

Moreover, it was observed that the milk powder formulation performed slightly better than the lactose tablets when comparing final CFU values. The reason for that might be the fact the milk is a rich medium with both buffer capacity and nutritional content.

### Shelf Life Testing

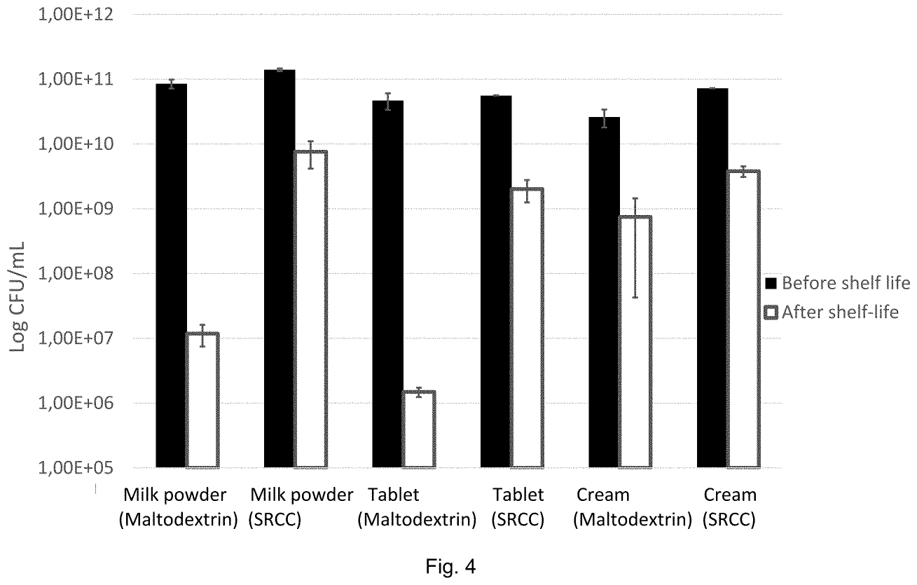

A CFU analysis of the stabilized samples was carried out according to ISO 1133-1:2009, before and after the shelf-life test, FIG. 4 shows the results of the accelerated shelf-life experiments. In line with the results of the in-vitro digestion testing, the inventive samples with SRCC outperformed the comparative samples with maltodextrin for the milk powder formulation and lactose tablets. Moreover, the cream formulation containing the inventive stabilizing agent SRCC performed slightly better than the cream formulation containing the comparative stabilizing agent maltodextrin.

In summary, it has been shown that the inventive stabilizing agent is an effective stabilizing agent in production (spray drying), delivery (shelf-life), and application (in-vitro digestion) of probiotic compositions. Moreover, the inventive stabilizing agent outperformed the comparative stabilizing agent maltodextrin.

1. A stabilized probiotic composition, comprising:
a stabilizing agent comprising a surface-reacted calcium carbonate; and
a probiotic microorganism culture;
wherein the surface-reacted calcium carbonate is a reaction product of a natural ground calcium carbonate or a precipitated calcium carbonate with carbon dioxide and one or more H₃O⁺ ion donors, wherein the carbon dioxide is formed in situ by the H₃O⁺ ion donors treatment and/or is supplied from an external source.

2. The stabilized probiotic composition of claim 1, wherein the probiotic microorganism culture is selected from the group consisting of Bifidobacterium adolescentis, Bifidobacterium lactis, Bifidobacterium infantis, Bifidobacterium longum, Bifidobacterium bifidum, Bifidobacterium breve, Lactobacillus acidophilus, Lactobacillus johnsonii, Lactobacillus casei, Lactobacillus reuteri, Lactobacillus rhamnosus, Lactobacillus plantarum, Lactococcus lactis, Enterococcus faecium, Escherichia coli Nissle 1917, Escherichia coli criodesiccata (083:K24:H31), Saccharomyces boulardii, Saccharomyces cerevisiae, and mixtures thereof.

3. The stabilized probiotic composition of claim 1, wherein the probiotic composition comprises a probiotic microorganism culture in an amount of at least 50 wt.-%, based on the total weight of the probiotic composition.

4. The stabilized probiotic composition of claim 1, wherein the stabilized probiotic composition is a dry composition or an aqueous suspension.

5. The stabilized probiotic composition of claim 1, wherein the surface-reacted calcium carbonate has one or more of:
i) a volume median particle size d₅₀ in the range of 0.1 to 75 μm;
ii) a volume top cut particle size d₉₈ in the range of 0.2 to 150 μm;
iii) a specific surface area in the range of 15 m²/g to 200 m²/g measured using nitrogen and the BET method; and
iv) an intra-particle intruded specific pore volume in the range of 0.1 to 2.3 cm³/g, calculated from mercury porosimetry measurement.

6. The stabilized probiotic composition of claim 1,
wherein the natural ground calcium carbonate is selected from the group consisting of marble, chalk, limestone, and mixtures thereof, or
the precipitated calcium carbonate is selected from the group consisting of precipitated calcium carbonates having an aragonitic, vateritic or calcitic crystal form, and mixtures thereof, and/or
the one or more H₃O⁺ ion donor is selected from the group consisting of hydrochloric acid, sulphuric acid, sulphurous acid, phosphoric acid, citric acid, oxalic acid, an acidic salt, acetic acid, formic acid, and mixtures thereof.

7. The stabilized probiotic composition of claim 1, wherein the weight ratio of probiotic microorganism culture:surface-reacted calcium carbonate is in the range of 5:95 to 40:60.

8. The stabilized probiotic composition of claim 1, wherein the stabilizing agent is a drying stabilizer and/or a shelf live preservative.

9. The stabilized probiotic composition of claim 1, wherein the stabilized probiotic composition is a pharmaceutical probiotic composition, a nutritional probiotic composition, or a cosmetic probiotic composition, and/or the stabilized probiotic composition is in the form of a tablet, a capsule, a chewable tablet, a chewable gum, a chewable pastille, a lozenge, a powder, a granulate, a pellet, a paste, a cream, a food, a feed, or a beverage.

10. The stabilized probiotic composition of claim 1, wherein the concentration of viable probiotic microorganism culture in the stabilized probiotic composition is at least 5% greater after drying the stabilized probiotic composition compared to a probiotic composition comprising maltodextrin as stabilizing agent.

11. A method for stabilizing a probiotic microorganism culture, comprising the step of mixing a probiotic microorganism culture with a surface-reacted calcium carbonate in an aqueous medium, wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source, and
drying the obtained mixture.

12. A process for preparing a dry stabilized probiotic composition, comprising the steps of:
a) providing an aqueous probiotic composition comprising at least 75 wt.-% of a probiotic microorganism culture, based on the total weight of the probiotic composition,
b) providing an aqueous suspension comprising 10 to 30 wt.-%, based on the total weight of the aqueous suspension, of surface-reacted calcium carbonate, wherein the surface-reacted calcium carbonate is a reaction product of natural ground calcium carbonate or precipitated calcium carbonate with carbon dioxide and one or more H₃O⁺ ion donors selected from the group consisting of hydrochloric acid, sulphuric acid, sulphurous acid, phosphoric acid, oxalic acid, or mixtures thereof, wherein the carbon dioxide is formed in situ by the H₃O⁺ ion donors treatment and/or is supplied from an external source,
wherein the surface-reacted calcium carbonate has a volume median particle size d₅₀ from 0.1 to 75 μm, and a volume top cut particle size d₉₈ from 0.2 to 150 μm, a specific surface area of from 15 m²/g to 200 m²/g, measured using nitrogen and the BET method, and
wherein the weight ratio of probiotic microorganism culture:surface-reacted calcium carbonate is from 5:95 to 40:60,
c) mixing the probiotic composition of step a) and the surface-reacted calcium carbonate of step b), and
d) spray drying the mixture obtained in step c), at an inlet temperature from 130 to 210° C. and an outlet temperature from 50 to 130° C.

13. A dry stabilized probiotic composition obtainable by the process according to claim 12.

14. A product comprising the dry stabilized probiotic composition according to claim 13, wherein the product is a tablet, a capsule, a chewable tablet, a chewable gum, a chewable pastille, a lozenge, a powder, a granulate, a pellet, a paste, a cream, a food, a feed, or a beverage.

15. The product according to claim 14, wherein the product is a pharmaceutical, nutritional or cosmetic.

16. The stabilized probiotic composition of claim 1, wherein the one or more H₃O⁺ ion donor is selected from the group consisting of hydrochloric acid, sulphuric acid, sulphurous acid, phosphoric acid, oxalic acid, H₂PO₄⁻, HPO₄²⁻, and mixtures thereof, wherein
wherein H₂PO₄₋ is at least partially neutralised by a cation selected from Li⁺, Na⁺ and/or K⁺; and
wherein HPO₄²⁻ is at least partially neutralised by a cation selected from Li⁺, Na⁺, K⁺, Mg²⁺, and/or Ca²⁺.
